(19) United States
(12) Patent Application Publication
(10) Pub. No.: US 2014/0303690 A2
(43) Pub. Date: Oct. 9, 2014

(54) ANTI-SLEEP GLASSES

(71) Applicants: Felice Eugenio Agro, Rome (IT); Luigi Agro, Rome (IT); Giuseppe Agro, Rome (IT); Francesco Agro, Rome (IT)

(72) Inventors: Felice Eugenio Agro, Rome (IT); Luigi Agro, Rome (IT); Giuseppe Agro, Rome (IT); Francesco Agro, Rome (IT)

(21) Appl. No.: 14/122,192

(22) PCT Filed: May 25, 2012
(86) PCT No.: PCT/EP2012/059896

§ 371 Date: Nov. 25, 2013

(30) Foreign Application Priority Data
May 26, 2011 (US) 61490352
May 25, 2012 (WO) PCT/EP2012/059896

(51) Int. Cl. A61N 1/36

(52) U.S. Cl. CPC A61N 1/36014
USPC 607/62

(57) ABSTRACT

A device for keeping awake a person that is about to fall asleep is proposed, comprising a pair of glasses with a frame that has two arms, at least one sensor for detecting the movements of an eye blink, at least one battery, and at least one electrode for issuing an electric pulse.

### BACKGROUND OF THE INVENTION

### Field Of The Invention

Many accidents are caused by loss of vehicle control due to drivers suddenly falling asleep. In most cases the driver is tired, and without realizing it, tends to temporarily close his eyes to rest, especially when the road is smooth and with little traffic, this momentary inattention can be fatal. Those who are most at risk are those people who drive for several hours for work-related reasons, especially during night time (e.g., truck drivers, bus drivers, taxi drivers, sales agents, etc.). Nevertheless there are currently no devices on the market able to recognize the onset of drivers “falling asleep” and prevent it, keeping the subject awake.

The aim of the next project is, therefore, to provide drivers with a device capable of keeping awake the person who is about to fall asleep.

Unlike previous devices, hereinafter referred to (*), the present invention does not use sounds or lights to awaken the driver, since often such systems have proved useless, but it is based on sending an electrical impulse to awake the subject.

### BRIEF DESCRIPTION OF THE RELATED ART

Many devices designed to wake up “sleepy” drivers have been filed at the patent office. However, they often proved impossible to carry out several problems: difficulty to wear it (too large, awkward or heavy), preventing and limiting movement or visibility, cannot be implemented mechanically or are bulky by having to use batteries or electricity connection for their operation. For example (*):

U.S. Pat. No. 5,689,241 entitled “Sleep detection and driver alert apparatus” and filed by Clarke Sr. et al. on Nov. 18, 1997 discloses a unit positioned around the rearview mirror which contains an infrared sensor that can detect changes in head positions and a temperature sensor. This sensor detects face temperature changes and, because during sleep a decrease of respiratory activity is detected, a decrease in the facial area temperature indicates an expiratory activity decrease, and therefore is indicative of sleep. According to this document, a sleep detection and driver alert apparatus with a compact housing is provided that can be placed around the rearview mirror or on the dashboard. It contains all lenses and electronic detection mechanisms for monitoring the effects of early impending sleep by means of an infrared auto-focusing, digital, image stabilizing lens with zoom capability. Additionally, the unit contains an added infrared thermal sensor for the monitoring and evaluation of different ambient temperatures around the facial areas of the nose and mouth. These temperature changes will be that of the exhaled gas plume of normal breathing patterns, which will lower in volume as the driver begins to hypoventilate, thus increasing their blood level of carbon dioxide which is in most part the reason for early drowsiness associated with sleep. The device will monitor via the infrared camera the thermal image changes in pixel color of open versus closed eyes of the driver via the temperature sensitive infrared portion of the digitized photographic image passed through a video charge coupling device. The combination of non-movement and a decrease in breath temperature, which is a physiological response to hypoventilation thus initiating drowsiness, will trigger the infrared camera to zoom onto the eye region of the driver. This combined data is routed to a sleep status microprocessor memory via the optical image detector and thermal sensor for data changes above or below baseline data measurements.

U.S. Pat. No. 5,402,109 entitled “Sleep prevention device for automobile drivers” and filed by Mannik on Mar. 28, 1995 discloses a device attached to the driver's glasses containing a beam of infrared light that detects movement of the eyelids and emits a noise when the eyelids are closed over a certain period. According to this document, a beam of narrow-band light of any color is used for optical sensing, whether the driver's eyelids are closed or are in an open position. The use of infrared light is preferred, because infrared light generates least distraction to the driver. A tiny slide-adjustable light emitter carrier, sliding along the eyeglass temple, is used for positioning the light emitter on the eyeglass properly for each driver. A narrow-band light beam from this emitter is aimed across the surface of the driver's eye, just above the eyeball, between the eyelids, and it is sensed in the opposite corner of the eye by means of a light sensor, which has a narrow band light filter mounted in front of it. For waking up the driver, whose eyes have been closed for a longer time period than about one second or less, an electronic circuitry is activated by means of the closed-eye signal from the light sensor, turning on an alarm signal, a buzzer or similar, after a one second or shorter time delay.

U.S. Pat. No. 5,469,143 entitled “Sleep awakening device for drivers of motor vehicles” and filed by Cooper on Nov. 21, 1995 discloses a device that consists of glasses containing a sensor lever placed in contact with one of the two upper eyelids of the driver. The downward movement of the eyelid also moves the lever activating the sensor that sends a command to a third unit to produce a bright flash. A pair of eyeglasses include a sensing lever that is in constant contact with one of the driver's upper eyelid muscles. Downward motion of the eyelid moves the sensing lever downward and actuates a microswitch that is coupled to the sensing lever. The microswitch in turn actuates circuitry located in the control unit to turn on a light each time the driver closes his eyelid. The circuitry includes a time delay relay that initiates an audible signal if the eyelid remains closed for a preset period of time. A normal blink of a driver's eye does not produce an audible alarm. However, if the driver's eyelid fails to open in a predetermined time, the audible alarm will sound. As soon as the driver's eyelid opens, a yellow caution light and the audible alarm are reset. Normal eye blinks produce illumination of the yellow caution light in view of the driver, thereby assuring the driver that the sleep awakening device is functioning properly.

In U.S. Pat. No. 4,875,030, entitled “Sleep-preventing alarm device” and filed by Chiu on Oct. 17, 1989 a LED positioned on a spectacle arm sends a beam of light to a sensor placed on the nose of the driver's glasses and when the eyelids are closed for an extended period of time the light beam is broken and a pulse generator emits a sound of such intensity as to wake the person. This sleep-preventing device incorporating within a pair of glasses has a light emitting diode, a photo diode, a buzzer, a power source and a printed circuit board. The printed circuit board is electrically connected to the light emitting diode, the photo diode and the buzzer. The photo diode is lodged in the bridge of the glasses and protrudes from a nosepiece of the glasses. An earpiece of the glasses has a cavity to receive the printed circuit board and the light emitting diode. An opening of the cavity is covered by a plate, and a cover on which the printed circuit board is disposed. The plate has a number of holes to receive the light emitting diode, in which bores of the holes are oriented towards the photo diode so that the photo diode receives the light from the light emitting diode.

U.S. Pat. No. 4,272,764 entitled “Self contained head mountable sleep inhibiting device” and filed by Herr et al. on Jun. 9, 1981 discloses a device placed on a hat or a pair of glasses, composed of a head movement sensor which sends a sound signal to wake the driver when they are altered, wherein a head mountable, audible alarm is disclosed, responsive to a nodding or tilting movement of the head of the user. The device as disclosed comprises a self contained head mountable sleep inhibiting device comprising a container having two interfitting parts for housing the electrical and mechanical components of the device and employing an L-shaped clamp member pivotally mounted at the intersection of the legs of the clamp on a corner of the housing. The clamp firmly grips the stem of glasses or the rim or band of a hat for ease in mounting the device on a user.

One of the major problem with all the devices as described above is that they are either too big to be useful, too cumbersome to use, or too complex to work reliably and efficiently.

Accordingly there is a need for devices that work reliably, and keep the user awake efficiently, while being small enough to be easy to handle and use that can be manufactured at a competitive price, and may be operated with as little energy consumption as possible.

### SUMMARY OF THE INVENTION

The device consists of a pair of glasses (with neutral, graduated or darkened lenses) on which a sensor, a battery and an electrode are implanted at the level of both arms.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

What is proposed is a device for keeping awake a person that is about to fall asleep, comprising a pair of glasses with a frame that has two arms, at least one sensor for detecting the movements of an eye blink, at least one battery, and at least one electrode for issuing an electric pulse.

Preferably, the at least one sensor, battery and electrode are implanted into the frame at the level of both arms of the pair of glasses, wherein it is even more preferred that the at least one electrode is located in an area of the arms of the pair of glasses, to enable a download on the parietal area of the skull of a wearer

Providing all the elements of the device within the regular frame of a pair of looking glasses make the device extremely easy to handle and to operate, as it can be handled exactly like any other pair of glasses. To this extend, the device according to the present invention may be provided with neutral, graduated or darkened lenses.

The sensor can detect the movements of the eye blink, considering the frequency of opening and closing of the upper eyelid. Several studies have shown that in a person who is about to fall asleep, the blinking tends to be more frequent, reaching a threshold of about 15-20 episodes per minute, while at the same time increasing the wink frequency with an eyelid closure duration higher than 0.03 msec.

These two variables can be altered or you can have both an increase of only one of the two parameters. In any case, the parameter that is more correlated with the risk of falling asleep is the increased eyelids closing time.

When these values reach a dangerous threshold the sensor is activated and sends a pulse to the battery. The battery, receiving the pulse from the sensor, sends the energy needed to the electrode to issue an electric pulse and thus cause an electric shock. This electrode preferably functions similarly to the electrodes of the TENS (Transcutaneous Electrical Nerve Stimulator) which are widely used in pain therapy, and give a download on the parietal area of the skull that is able to awaken the driver, making it possible for him to reach the nearest resting point. These effects are described for example in, L. S. Chesterton, N. E. Foster, C. C. Wright, G. D. Baxter, and P. Barlas, “Effects of TENS frequency, intensity and stimulation site parameter manipulation on pressure pain thresholds in healthy human subjects,” Pain 106:73-80 (2003) and Josimari M. DeSantana, Deirdre M. Walsh, Carol Vance, Barbara A. Rakel, and Kathleen A. Sluka, “Effectiveness of Transcutaneous Electrical Nerve Stimulation for Treatment of Hyperalgesia and Pain,” Curr Rheumatol Rep. 2008 December; 10(6): 492-499.

In any field the TENS is used, the most important moment is the choice of frequency and intensity of the pulse to be sent. The pulse frequencies that are preferred for the present invention and that are considered to be safe are between 2 Hz and 140 Hz, and may be varied depending on the type of pain to treat. The preferred maximum pulse for the present invention is 80 mA.

Further features, elements and advantages may be taken from the following, strictly non-limiting, description of a preferred embodiment of the present invention with reference to the drawing enclosed herewith; showing in:

### BRIEF DESCRIPTION OF THE DRAWINGS

FIG. 1 a schematic three-dimensional representation of a device according to the present invention.

### DETAILED DESCRIPTION OF THE PREFERRED EMBODIMENTS

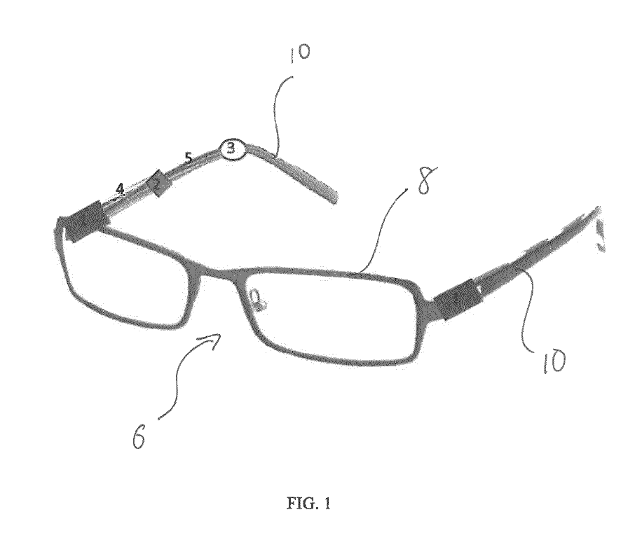

In FIG. 1 a preferred embodiment of the device according to the present invention is shown, comprising a pair of glasses 6, with a frame 8 and two arms 10 attached thereto.

What can be taken from FIG. 1 furthermore, are two sensors 1, a batterie 2, a TENS electrode 3, and connecting devices 4 and 5, connecting the battery with the sensor 1 on the one hand, and connecting the battery with the electrodes 3 on the other hand.

It should be noted that due to clarity reasons, only one battery 2 and one electrode 3 are shown, while in the actual embodiment there are batteries and electrodes in both arms 10.

The frame in this embodiment is provided with neutral lenses.

The TENS sensor 1 is designed to detect the movements of the eye blink, considering the frequency of opening and closing of the upper eyelid.

When these values reach a dangerous threshold, the sensor 1 is activated and sends a pulse to the battery 2. The battery 2, receiving the pulse from the sensor 1, then sends the energy needed to the electrode 3 to issue an electric pulse and thus cause an electric shock.

The pulse frequencies that the electrodes 3 operate at in the preferred embodiment are between 2 Hz and 140 Hz, while the maximum pulse for the embodiment as shown here is 80 mA.

The electrodes 3 used in the embodiment as shown, are capable of imparting low frequency pulses and high intensity.

1. Device for keeping awake a person that is about to fall asleep, comprising a pair of glasses with a frame that has two arms, at least one sensor for detecting the movements of an eye blink, at least one battery, and at least one electrode for issuing an electric pulse.

2. Device according to claim 1, wherein the at least one sensor, battery and electrode are implanted into the frame at the level of both arms.

3. Device according to claim 1 or 2, wherein the pair of glasses is provided with neutral, graduated or darkened lenses.

4. Device according to any of the preceding claims, wherein the at least one electrode is a TENS (Transcutaneous Electrical Nerve Stimulator) electrode.

5. Device according to any of the preceding claims, wherein the at least one electrode is located in an area of the arms of the pair of glasses, to enable a download on the parietal area of the skull of a wearer.

6. Device according to any of the preceding claims, wherein the at least one electrode is designed to issue a pulse between 2 Hz and 140 Hz.

7. Device according to claim 6, wherein the at least one electrode is capable of varying the pulse frequency.

8. Device according to any of the preceding claims, wherein the at least one electrode issues a maximum pulse of 80 mA.
